(19) United States
(12) Patent Application Publication
(10) Pub. No.: US 2012/0058009 A1
(43) Pub. Date: Mar. 8, 2012

(54) ANALYZER

(75) Inventors: Makoto Nogami, Tsuchiura (JP); Yuichiro Hashimoto, Tachikawa (JP); Izumi Waki, Tokyo (JP); Katsuhiro Kanda, Hitachinaka (JP)

(73) Assignee: HITACHI HIGH-TECHNOLOGIES CORPORATION, Tokyo (JP)

(21) Appl. No.: 13/201,659

(22) PCT Filed: Jan. 18, 2010
(86) PCT No.: PCT/JP2010/000215

§ 371 Date: Sep. 13, 2011

(30) Foreign Application Priority Data
Mar. 5, 2009 (JP) 2009-051457

(51) Int. Cl. G01N 33/48

(52) U.S. Cl. 422/63

(57) ABSTRACT

An analyzer includes a pretreatment device and a mass spectrometer. The pretreatment device includes a solid phase extraction mechanism. The mass spectrometer performs mass spectrometry on a sample pretreated by the pretreatment device and then subjected to ionization. The analyzer also includes a storage unit that stores data on dependence of signal intensities of the analyte substance to be measured and the internal standard upon the concentration of a substance inhibiting ionization in the sample, and that stores data on a recovery rate. The analyzer also includes a correcting unit that corrects measurement results of the sample and the internal standard on the basis of the data stored in the storage unit.

### TECHNICAL FIELD

The present invention relates to an analyzer that analyzes a biological sample such as blood using mass spectrometry, and more particularly to an analyzer that is equipped with a pretreatment device that performs a pretreatment such as a solid phase extraction.

### BACKGROUND ART

Immunoassay method is one of analytical methods that are widely used for clinical analysis. In the immunoassay method, an antibody (antigen) specifically recognizes an analyte component to be measured contained in a sample. For example, in an immunoassay method, after an analyte component to be measured, which is contained in a liquid, is captured by an antibody (primary antibody), a secondary antibody that selectively attaches to the primary-antibody analyte complex is used for detection. In this case, a label is added to the secondary antibody to detect the secondary antibody with high sensitivity. The label for the secondary antibody may be a fluorescent substance, a substance required for enzyme chemiluminescence, or the like, for instance. The immunoassay method is a technique that enables a simple and high-sensitivity detection. Thus, the immunoassay method is suitable for a quantitative measurement of a low-concentration component contained in a specimen. In contrast, the immunoassay method has a problem with cross reactivity. The cross reactivity is an effect in which a primary antibody captures not only an analyte component to be recognized and measured but also a molecule (such as a metabolite of the component to be measured) having a structure similar to that of the component to be recognized and measured. This means that the result of the quantitative measurement is larger than a true value and the component to be measured cannot be accurately quantitated. Especially, when the component to be measured is a low-molecular compound, the cross reactivity tends to be noticeable. To develop an antibody for small molecules, it is necessary to attach a carrier protein to the analyte component. Only a part other than a part to which the carrier protein is attached to may become an epitope. This is one of the reasons that the difference between a structure of the metabolite and a structure of the analyte component to be measured cannot be identified. To suppress the cross reactivity, a primary antibody that can identify differences between various molecules having similar structures needs to be formed. It is, however, difficult to form such a primary antibody. In addition, the cost and efforts are increased, and it is not efficient to form such a primary antibody.

Comparing to the immunoassay method, in a mass spectrometry method, an analyte component is measured on the basis of the mass of the component to be measured. Thus, the mass spectrometry is a measurement technique capable of identifying a molecule (such as a metabolite) having a similar structure. Especially, in an MS/MS analysis and an MSn analysis, a component to be measured is converted into a fragment signal. Thus, the MS/MS analysis and the MSn analysis are techniques capable of identifying components having similar structures with high accuracy. The mass spectrometry method is superior in selectivity and accuracy than the immunoassay method. Thus, there is a trend that the mass spectrometry method is increasingly introduced for clinical use.

In Patent Document 1, dried blood is treated as a specimen, and an analyte component to be measured is extracted by a liquid-liquid extraction and measured by a mass spectrometer. Then, amino acids (such as alanine and valine) and acylcarnitines are quantified, and metabolic disorders are screened by testing the degree of a metabolic reaction involving the analyte substance in a living body. As an MS mode, a multiple reaction monitoring (MRM) mode of a triple quadrupole mass spectrometer having high selectivity is used. MRM is a method for passing only a precursor signal by means of a quadrupole located at the first stage, fragmenting the signal in the next collision cell, and monitoring only a product signal specific to a generated compound by means of a second quadrupole. In this method, the analyte substance to be measured can be identified using mass information specific to the compound. In addition, by using the mass information, the component to be measured can be separated by the mass number from an impurity contained in the specimen. In a quantitative method, standard substances are first analyzed in a conventional manner at several concentrations, where one calculates an area of a peak in each mass chromatogram, which is a temporal response curve of the intensity of a signal for a mass-to-charge ratio m/z corresponding to the standard substances. A calibration curve is formed on the basis of the relationship between the peak areas and the concentrations of the standard substances. Next, the same substance with unknown concentration is analyzed, where a peak area of the mass chromatograph is calculated. One converts the peak area into the concentration of the substance using the formed calibration curve. An internal standard is used for a correction of data obtained in the entire measurement. A stable-isotope-substituted analyte molecule is used as the internal standard for each analyte to be measured. The internal standard is added to an eluting solution to be used for a liquid-liquid extraction of dried blood. In Non-Patent Document 1, an organic solvent is added to a specimen, and a protein is precipitated. After that, the specimen is introduced into a mass spectrometer after separation, using a liquid chromatography/mass spectrometry (LC/MS) device. Then, 12 types of hormones (such as estradiol and testosterone), which are contained in a living body at low concentrations, are simultaneously detected. A stable-isotope-substituted analyte molecule is used as an internal standard for each of substances to be measured. The internal standard is added to a buffer solution to be used for precipitation of a protein. In Patent Document 2, a biological sample such as a serum or urine is treated using a solid phase extraction plate having 96-wells and measured by a mass spectrometer so that amino acids, carnitines, sugars, an immunosuppressants and the like are quantitated. As the internal standard, a stable-isotope-substituted analyte molecule or an analog molecule having a similar chemical structure can be used. The internal standard is added to a buffer solution to be used for precipitation of a protein.

When the mass spectrometry method is used for clinical application, ion suppression, which is specific to the mass spectrometry method, is a problem. Ion suppression can affect the accuracy of quantitation, because it can inhibit the efficiency of converting an analyte component into a measurable signal in the mass spectrometry method. Thus, a stable-isotope-substituted analyte molecule, which can be regarded to cause the same level of ion suppression, is normally used. In the mass spectrometry method, components to be measured can be measured at time intervals of several milliseconds. Thus, multiple components can be quantitated with high throughput. However, one requires as many kinds of stable-isotope-substituted analyte molecules as the number of the analyte components. It is costly to synthesize the stable-isotope-substituted analyte molecules. A further problem is that a stable-isotope-substituted analyte molecule cannot be formed for a component that cannot be synthesized. As described in Patent Document 2, an analog molecule is used as the internal standard in some cases. In this case, an effect of a matrix component (impurity) on the analyte component to be measured is not equivalent to an effect of the matrix component on the internal standard. Therefore, even if a correction is performed, it is not ensured that an accurate value can be obtained. When an analog molecule is used as the internal standard, pretreatments such as LC and solid phase extraction processes are performed to separate the matrix component from the analyte substance to be measured, so as to reduce an effect of the matrix. In Non-Patent Document 2, LC conditions are optimized so that an analyte component enters a mass spectrometer when a matrix is not introduced into the mass spectrometer. The optimization is achieved using a post column infusion method, whereby a matrix component after LC separation is continuously introduced into a mass spectrometer, while an analyte component to be measured is infused continuously through a different path, so as to identify in advance the time interval during which an effect of the matrix is large. However, according to the aforementioned methods, times and costs are increased, and the operations are complicated.

When the mass spectrometry method is used for a clinical application, it is necessary that data do not vary depending on a facility and a user of the instrument. In other words, it is important to provide a mass spectrometer that is simple and easy to use as much as possible. Specifically, it is desirable that manual operations be eliminated as much as possible in a pretreatment, a MS measurement and a data analysis, and the mass spectrometer be capable of performing a fully automatic analysis. In this case, the stability and a recovery rate of the pretreatment process significantly affect the accuracy of data, to a similar extent as the effect of the ion suppression.

### PRIOR ART REFERENCES

### Patent Documents

Patent Document 1: US2006/0008922 A1

Patent Document 2: US2007/0004044 A1

### Non-Patent Documents

Non-Patent Document 1: T. Guo, R. L. Taylor, R. J. Singh, S. L. Soldin, Simultaneous determination of 12 steroids by isotope dilution liquid chromatography-photospray ionization tandem mass spectrometry, Clinica. Chemica. Acta., 372, 76-82, 2006

Non-Patent Document 2: T. M. Annesley, Ion Suppression in Mass Spectrometry, Clin. Chem. 49:7, 1041-1044, 2003

### SUMMARY OF THE INVENTION

### Problems to be Solved by the Invention

When a quantitative analysis is performed using a mass spectrometer, an internal standard method is used. A stable-isotope-substituted analyte molecule that has a property same as or similar to that of an analyte substance to be measured is ideally used as an internal standard to perform a correction. When the mass spectrometer is used, ion suppression that is caused by an impurity contained in a matrix is noticeable and affects the accuracy in data, unlike a spectrophotometer and a UV detector. When an analog molecule is used as the internal standard instead of the stable-isotope-substituted analyte molecule, sufficient separation and refinement are performed by LC and GC so as to avoid ion suppression due to the impurity contained in the matrix. In addition, the impurity affects the accuracy in data obtained in a pretreatment performed on a sample, in addition to the ion suppression. Thus, reproducibility of the data is low compared to other measurement methods. In a conventional method, when a quantitative analysis is performed using a mass spectrometer, a calibration curve is formed for each of measurements and the concentration of an analyte substance to be measured in a sample is calculated using the internal standard method. Thus, when a stable-isotope-substituted analyte molecule is used for the internal standard, there is a problem with a cost. In contrast, when an analog molecule is used for the internal standard, there is a problem with an effort for separation and purification, the cost of a eluent and throughput. In addition, since it is necessary to form a calibration curve for each of measurements, there is a problem with the cost of a reagent and throughput.

### Means for Solving the Problems

According to the present invention, a simple and practical method for correcting data has been devised on the basis of the fact that an impurity contained in a matrix affects the accuracy in data obtained by a mass spectrometer, while addressing the issue of a cost and throughput. Specifically, an accurate correction can be performed on a sample containing multiple analyte substances to be measured using at least one analog molecule, without using individual internal standards. A lot of specimens can be simultaneously processed in parallel and analytical test results can be obtained by providing a pretreatment device, pretreated sample transporting means 109, a mass spectrometer 111 that is capable of fully automatically performing an analytical measurement, and a data processing unit 112. The pretreatment device includes: a solid phase extraction cartridge 101 into which a liquid to be analyzed is introduced so that a specific component is selectively separated; a cartridge holding vessel 102 that holds a solid phase extraction cartridge 101 therein; cartridge transporting means 103 that is capable of holding a plurality of storage sections (cartridge holding vessels 102) and has a continuous-track; a pressure applying unit 107 that can access the storage sections in a random order, and that can continuously apply pressure to an inside of a storage section; and an extracted solution receiving mechanism 108 that selects a storage section where the receiving mechanism receives a solution extracted from the separating agent stored in the storage section.

For a correction using an internal standard, an analyte substance to be measured with a known concentration and the internal standard with a known concentration are added to a matrix having a different physical property in advance, and the data processing unit 112 then stores data on correlations between the property of the matrix and the sensitivity. The sensitivity is a value obtained by dividing an intensity of a measurement signal by a concentration. In addition, data on a recovery rate of the analyte substance to be measured for each of properties of the matrix is stored as a recovery rate in a pretreatment such as a solid phase extraction. Next, one measures a specimen which contains the internal standard with known concentration and the analyte substance to be measured with unknown concentration. A value of the property of the matrix is calculated from the actual measured intensity of a signal of the internal standard. The internal standard is dispensed in the extracted solution receiving mechanism 108 by a rotating arm 105 before the internal standard is transported to the mass spectrometer 111 by the pretreated sample transporting means 109. The data processing unit 112 is accessed when the intensity of the signal of the internal standard with the known concentration is measured by the mass spectrometer 111. Then, a value of each of the properties of the matrix is calculated from data stored in the data processing unit 112. After the analyte substance to be measured is measured by the mass spectrometer 111 so that the intensity of a signal is obtained, the data processing unit 112 is accessed, and the concentration of the analyte substance to be measured is calculated from the value, calculated from the stored data, of each of the properties of the matrix and the intensity of the signal of the analyte substance to be measured. Parameters of the properties of the specimen are a phospholipid concentration, a viscosity (density, dilution ratio), pH and the total mass of proteins, for example. Lastly, the concentration of the analyte substance to be measured is calculated by reflecting the recovery rate of the analyte substance to be measured for each of the properties of the matrix during the pretreatment process. The concentration of the analyte substance to be measured can be calculated using the internal standard with the known concentration on the basis of the correlation data stored in the data processing unit 112. When this correction method is used, multiple analyte components to be measured can be corrected using only one internal standard without forming a calibration curve in principle.

### Effect of the Invention

According to the present invention, ion suppression and a recovery rate in the pretreatment such as the solid phase extraction that are problematically not corrected in the conventional method, can be simply and accurately corrected. It is possible to correct data on a sample containing multiple analyte substances to be measured without using internal standards for every analyte substances to be measured. In addition, it is not necessary to prepare several analyte substances of known concentrations and several stable-isotope-substituted analyte molecules of known concentrations and to form a calibration curve, which improves the efficiency of time and cost. Thus, a pretreatment in which a complicated operation is performed can be omitted. Therefore, the configuration of the analyzer can be simplified, and a simple and highly accurate clinical analyzer can be achieved.

### BRIEF DESCRIPTION OF THE DRAWINGS

FIG. 1 is an outline diagram illustrating a configuration of an analyzer.

FIG. 2 is an outline diagram illustrating the flow of measurement.

FIG. 3 is an outline diagram illustrating a correction method.

FIG. 4 is a conceptual diagram illustrating dependence (sensitivity) of signal intensity with respect to a value relating to a property of a matrix.

FIG. 5 is a conceptual diagram illustrating recovery rate with respect to a value relating to a property of a matrix.

FIG. 6 is an outline diagram illustrating the flow of measurement.

FIG. 7 is an outline diagram illustrating a correction method.

FIG. 8 is an outline diagram illustrating a value input to a data processing unit and a value output from the data processing unit.

FIG. 9 is a conceptual diagram illustrating dependence (sensitivity) of signal intensity with respect to the intensity of signal of lecithin.

FIG. 10 is a conceptual diagram illustrating recovery rate with respect to the intensity of signal of lecithin.

### MODE FOR CARRYING OUT THE INVENTION

Embodiments of the present invention are described in detail with reference to the accompanying drawings. However, the present invention is not limited to the following embodiments.

### First Embodiment

A purpose of a clinical application using mass spectrometry is to perform therapeutic drug monitoring (TDM). For example, in TDM, a pharmacokinetics of a drug is monitored. Before a medical drug is administered to a patient in a medical site, it is important to construct an individualized dosing plan for each patients on the basis of symptoms of each patient to ensure effectiveness and safety of a drug. As a cause of a variation in therapeutic effects depending on patients even when the patients take the same amount of drug, the concentrations of substances in blood vary due to differences among the individuals in terms of pharmacokinetics. Thus, TDM is performed to optimize the amount and interval of a drug dosing, so as to control the therapeutic drug concentration to fall in a effective therapeutic range, by measuring the concentrations of drugs in the blood of the patients. An example of drugs for which TDM is required are immunosuppressant drugs, which are used to suppress a rejection response to a transplanted organ. The concentration of a immunosuppressant in blood is typical low, in the range of (therapeutic range) of several nanograms/mL to several hundred nanograms/mL. When the concentration of an immunosuppressant drug in blood exceeds the therapeutic range, the immunosuppressant may cause a serious side effect such as hypertension, dyslipemia, hyperglycemia, peptic ulcer, or dysfunction of the liver or kidney. To reduce the side effects, cocktail administration may be performed in general. Or, several types of immunosuppressants may be given in combination with steroids. It is difficult to chemically synthesize some immunosuppressants. Thus, a stable-isotope-substituted analyte molecule that is used as an internal standard may not exist. Therefore, an analog molecule is often used as an internal standard.

The present embodiment describes an example of an analyzer that performs a solid phase extraction as a pretreatment and uses a mass spectrometer as a detecting unit.

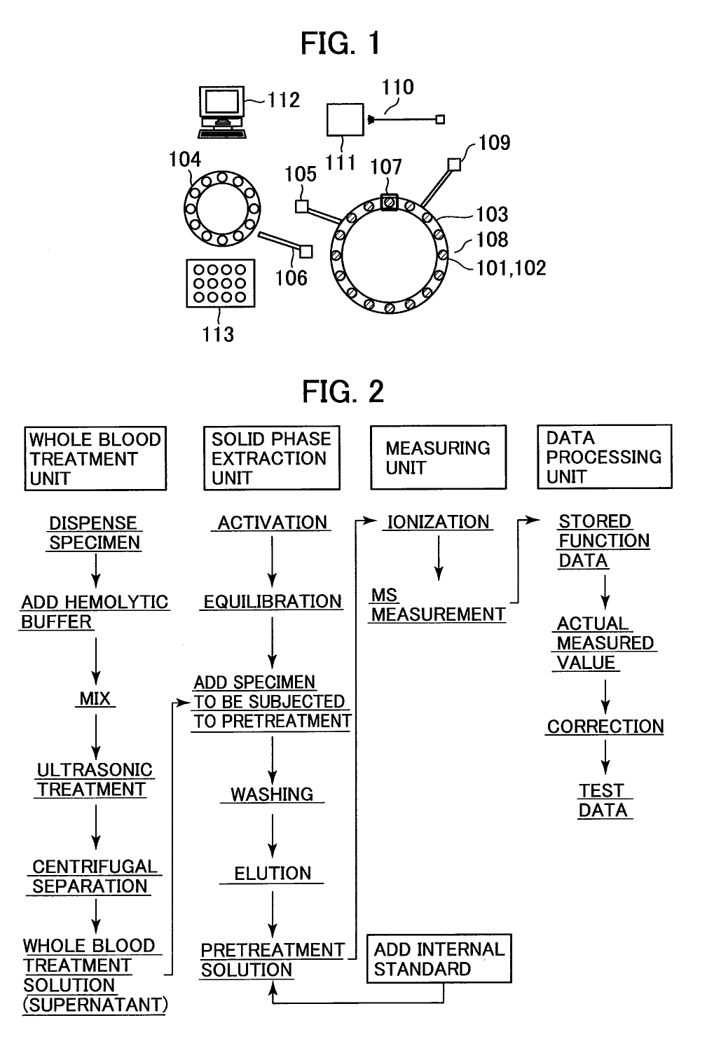

The configuration of the analyzer is described with reference to FIG. 1. The analyzer includes a pretreatment device. The pretreatment device includes: a solid phase extraction cartridge 101 that causes a solution (to be inspected) to be introduced in the cartridge 101 and can selectively separate a specific component; a cartridge holding vessel 102 that holds a solid phase extraction cartridge 101 therein; cartridge transporting means 103 that is capable of holding a plurality of storage sections and has a continuous-track; a whole blood treatment unit 113 that is capable of performing a refining treatment on whole blood; a reagent container 104 that is capable of storing a plurality of reagents; a rotating arm 105 that is capable of transporting a reagent from the reagent container to the solid phase extraction cartridge; a rotating arm 106 that is capable of transporting a reagent from the reagent container to the whole blood treatment unit; a pressure applying unit 107 that can access the storage sections in a random order, and that can continuously apply pressure to the an inside of a storage section; and an extracted solution receiving mechanism 108 that selects a storage section where the receiving mechanism receives a solution extracted from the separating agent stored in the storage sections. In addition, the analyzer includes pretreated sample transporting means 109; an ionizer 110 that ionizes a sample and causes the sample to be introduced into a mass spectrometer; the mass spectrometer 111 that is capable of analyzing and measuring the sample; and a data processing unit 112. In the present embodiment, a triple quadrupole mass spectrometer (Triple QMS) is used as the mass spectrometer 111. As the mass spectrometer 111, a single quadrupole mass spectrometer (QMS), a time-of-flight mass spectrometer (TOFMS), an ion trap mass spectrometer (ITMS) and a Fourier transform ion cyclotron resonance mass spectrometer (FT-ICRMS) can be used.

The flow of a measurement is described with reference to FIG. 2. A patient's specimen of 100 μL, to whom an immunosuppressant is administered, is dispensed in the whole blood treatment unit 113. A methanol of 200 μL (stored in the reagent container 104) that contains 0.2M zinc sulfate is dispensed in each of cells of the whole blood treatment unit 113 by the rotating arm 106. The whole blood is fully miscible with the methanol of 200 μL that contains 0.2M zinc sulfate by application of an ultrasonic wave for approximately one minute, where the whole blood treatment unit 113 has an ultrasonic generating function. Thus, cell membranes of red blood cells are disintegrated (hemolyzed). In this case, the efficiency of the hemolysis is significantly improved by the application of the ultrasonic wave at a high temperature of 70° C. to 80° C. The hemolysis can be completed for a short time of approximately 10 seconds. The addition of the zinc sulfate causes a deproteination effect. Next, centrifugal separation is performed. A supernatant of approximately 200 μL is dispensed in the solid phase extraction cartridge 101 by the rotating arm 106. The immunosuppressant has a property to transfer into a blood cell, and the hemolysis operation is therefore required. In contrast, the hemolysis operation is not necessary for other therapeutic drugs that do not have a blood cell transfer property, such as, for example, an antiepileptic drug and an antimicrobial agent. For these drugs, the patient's specimen of 100 μL, which is dispensed in the whole blood treatment unit 113, is separated by centrifugal separation. Then, a serum or a plasma component, which is a supernatant, is dispensed in the solid phase extraction cartridge 101 by the rotating arm 106.

Before the treated solution after the hemolysis operation is dispensed in the solid phase extraction cartridge 101, activation and equilibration are performed on the solid phase extraction cartridge 101. In this example, a 100% methanol solution of 200 μL, which is stored in the reagent container 104, is dispensed in the solid phase extraction cartridge 101 by the rotating arm 105. The 100% methanol solution is transported by the cartridge transporting means 103 to a location at which at least one pressure applying unit 107 exists. Then, pressure is applied to the methanol solution, and introduced in the cartridge. Thereby, the separating agent is activated. Next, a 100% distillated water of 200 μL, which is stored in the reagent container 104, is dispensed in the solid phase extraction cartridge 101 by the rotating arm 105. Then, the 100% distillated water is transported to the pressure applying unit 107 by the cartridge transporting means 103. Then, pressure is applied to the distillated water, and introduced in the cartridge. Thereby, the separating agent is equilibrated. After that, the treated solution after the hemolysis operation is dispensed in the solid phase extraction cartridge 101 by the rotating arm 106 and pressure is applied thereto. An analyte substance to be measured and the internal standard are therefore captured by the separating agent. Next, cleaning is performed by causing the 100% distillated water of 200 μL to be introduced in the cartridge. Lastly, the 100% methanol solution of 200 μL is introduced in the cartridge, and the treated solution that has been extracted by the solid phase extraction is eluted into the extracted solution receiving mechanism 108. Then, the treated solution is transported by the pretreated sample transporting means 109 and ionized by the ionizer 110. Thereafter, the treated solution is introduced into the mass spectrometer 111, and the measurement is then performed. Before the treated solution is transported by the pretreated sample transporting means 109, the internal standard stored in the reagent container 104 is dispensed in the extracted solution receiving mechanism 108 by the rotating arm 105. The internal standard is then added to the treated solution. In addition, the composition of a solvent that is used for the cleaning process and the elution process varies depending on the combination of the internal standard and the analyte substance to be measured. To purify from an impurity as much as possible during the solid phase extraction process, a 40% methanol solution is used in the cleaning process, and a 90% methanol solution is used in the elution process in some cases. As the purifying means, a liquid-liquid extraction, deproteinization, an ultrafiltration membrane and antibody magnetic beads can be used as well as the solid phase extraction.

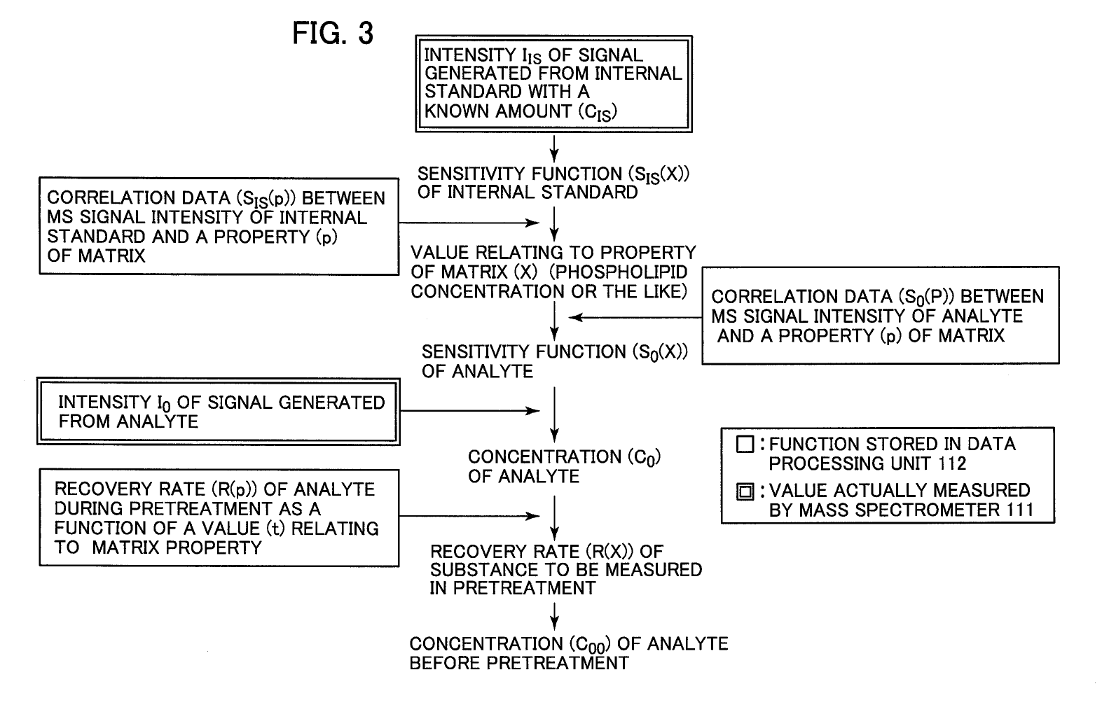

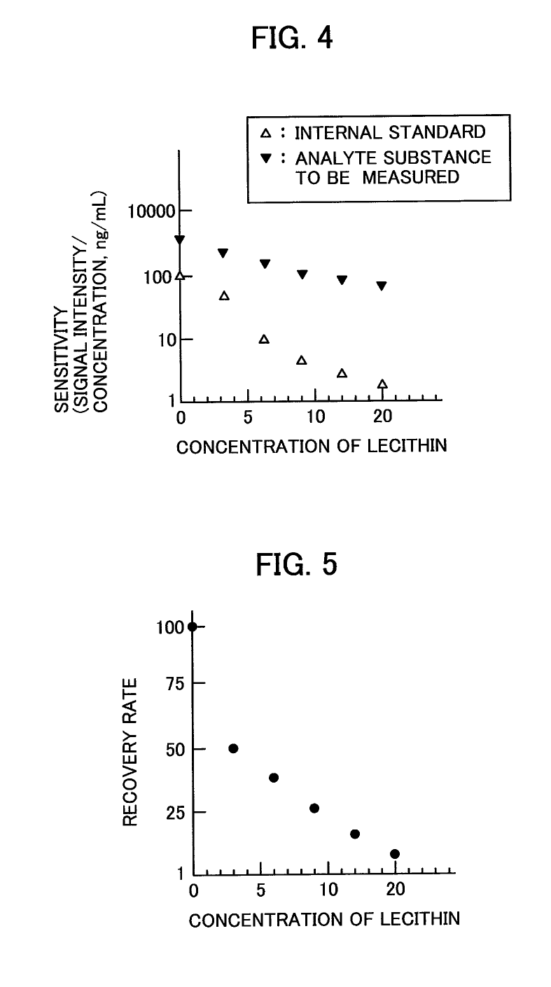

A correction method is described below. For an analyzer that uses the solid phase extraction in the pretreatment and uses a MS for detection, pretreatment recovery rate and ion suppression significantly affect the reproducibility and the accuracy of the data. In general, a calibration curve is formed by measuring specimens using MS detection wherein an internal standard and the analyte substance to be measured with known several concentrations are added to the specimen before the pretreatment. Here, the concentration of the analyte substance to be measured is plotted along the abscissa, whereas plotted along the ordinate is a ratio of the intensity of a signal corresponding to the mass-to-charge ratio m/z of the analyte substance to the intensity of a signal corresponding to the mass-to-charge ratio m/z of the internal standard. Then, the patient's specimen is actually measured, and the concentration of the analyte substance to be measured is calculated on the basis of the constructed calibration curve and the ratio of the intensity of the signal corresponding to the mass-to-charge ratio m/z of the analyte substance to the intensity of the signal corresponding to the mass-to-charge ratio m/z of the internal standard. In this method, the cost of the reagent is considerable, and the time for the formation of the calibration curve is long. Therefore, acquisition of the calibration curve is complicated. Also, because it is necessary that ion suppression of the internal standard be equivalent with ion suppression of the analyte substance to be measured, the internal standard requires a stable-isotope-substituted analyte molecule for the analyte substance to be measured. A correction method according to the present invention is described with reference to FIG. 3. The analyte substance to be measured with a known concentration and the internal standard with a known concentration are added to matrices having different properties. Correlation data (FIG. 4) is stored in the data processing unit 112. In the correlation data, a value relating to the property of the matrix is plotted along the abscissa; plotted along the ordinate is the dependence (sensitivity) of the intensities of the signals that corresponds to the mass-to-charge ratios m/z of both the analyte substance to be measured and the internal standard as a function of the value relating to the property of the matrix. The sensitivity is a value obtained by dividing an intensity of the signal corresponding to the mass-to-charge ratios m/z by the concentration. A value relating to the property of the matrix is, for example, the concentration of phospholipid. Examples of phospholipids are glycerophospholipids and sphingophospholipids. In the present embodiment, lecithin (phosphatidylcholine) which is a glycerophospholipid is added to the matrix. For other phospholipids, lysolecithin and cephalin (phosphatidylethanolamine) may be considered as glycerophospholipids, while sphingomyelin is considered as a sphingophospholipid. As values relating to the property of the matrix, a viscosity, the total mass of proteins and pH are used, as well as the aforementioned phospholipids concentration. In this case, the concentration p of phospholipid lecithin is a variable value relating to the property of the matrix. That is, a sensitivity function S₀(p) of the analyte substance to be measured and a sensitivity function S_{IS}(P) of the internal standard are stored in the data processing unit 112 in this case The intensity (actually measured by the mass spectrometer 111) of the signal of the analyte substance to be measured is indicated by I_{O}, while the intensity (actually measured by the mass spectrometer 111) of the signal of the internal standard is indicated by I_{IS}. When the value relating to the property of the matrix contained in the analyte substance to be measured is indicated by X, the concentration of the internal standard is indicated by C_{IS}, and the obtained concentration of the analyte substance to be measured is indicated by C₀, sensitivity S_{IS}(X) of a standard compound is expressed by Equation (1).

S_{IS}(X)=I_{IS}/C_{IS} (1)

The value X relating to the property of the matrix can be calculated from the result obtained from Equation (1) and the function S_{IS}(p) stored in a database beforehand. Next, the sensitivity S₀(X) of the analyte substance to be measured is calculated from the value X relating to the property of the matrix and the function S₀(p) stored in the data processing unit 112. The concentration C₀ of the analyte substance to be measured can be calculated according to Equation (2) on the basis of the sensitivity S₀(X) and the intensity I₀ (actually measured by the mass spectrometer 111) of the signal of the analyte substance to be measured.

C₀=I₀/S₀(X) (2)

The ion suppression can be corrected by the aforementioned operations. The value relating to the property of the matrix is used as a variable for each of the sensitivity functions of the analyte substance to be measured and the internal standard. As the sensitivity function (stored in the data processing unit 112) of the analyte substance to be measured and the sensitivity function (stored in the data processing unit 112) of the internal standard, a multi-dimensional sensitivity function of the analyte substance to be measured and a multi-dimensional sensitivity function of the internal standard can be used to improve the accuracy of the correction, where multiple properties of the matrix, that affect the ion suppression, are used as multiple variables in the multi-dimensional sensitivity functions.

Next, a correction of the recovery rate in the pretreatment is described. A recovery rate R(p) of the analyte substance to be measured is stored in the data processing unit 112 beforehand, while the concentration p of the phospholipid is used as a variable for the recovery rate R(p). The recovery rate R(p) is a value obtained by dividing the intensity of a signal of an analyte substance after pretreatment by that before treatment; whereby, an analyte signal after pretreatment is obtained by adding the analyte substance to be measured to the matrix containing the phospholipid, then by subjecting it to the pretreatment with a resultant concentration p of the phospholipid after the pretreatment, and further subjecting it to the MS detection; and whereby, an analyte signal before pretreatment is obtained by adding the analyte substance to be measured to the matrix containing the phospholipid with the concentration p, and directly subjecting it to the MS detection. The concentration C₀₀ of the analyte substance to be measured before the pretreatment is calculated according to Equation (3). The value X relating to the property of the matrix can be calculated from the result obtained from Equation (1) and the function S_{IS}(p) stored in the database.

C_{OO}=C_{O}/R(X) (3)

The recovery rate R(p) varies depending on the phospholipid concentration and other values (such as phospholipid other than lecithin, the viscosity, the total mass of protein and pH) relating to properties of the matrix. In addition, the recovery rate R(p) also varies depending on the type of the separating agent of the solid phase extraction cartridge 101. In these cases, the function of the recovery rate R_{X}(p) is stored in the data processing unit 112.

In this manner, using the internal standard with a known concentration that is added to the specimen, the concentration of the analyte substance to be measured can be obtained from the correlation data stored in the data processing unit 112. In this correction method, a plurality of substances to be measured can be corrected using, in principle, a single internal standard without forming a calibration curve.

### Second Embodiment

Regarding the correction method, a method for performing a correction feedback while measuring a material different in property of the matrix in real time, is described below. The configuration of the analyzer and the flow of the measurement are the same as those of the first embodiment. A correction method that is different from the correction method according to the first embodiment is described. In the first embodiment, the analyte substance to be measured with a known concentration and the internal standard with a known concentration are added to the matrices having different properties. In the first embodiment, the correlation data is stored in the data processing unit 112. In the correlation data, the value relating to the property of the matrix is plotted along the abscissa, while the dependence (sensitivity) of the intensities of the signals that correspond to the mass-to-charge ratios m/z of the analyte substance to be measured and the internal standard as a function of the value relating to the property of the matrix, is plotted along the ordinate. The value relating to the property of the matrix is calculated from the intensity (actually measured by the mass spectrometer 111) of the signal of the internal standard (with the known concentration) added to the specimen. Next, the concentration of the analyte substance to be measured is calculated from the actual measured intensity of the signal of the analyte substance to be measured and the value relating to the property of the matrix. Then, the concentration of the analyte substance to be measured before the pretreatment is calculated from the recovery rate (stored in the data processing unit 112) of the analyte substance to be measured in the pretreatment, while the value relating to the property of the matrix is used as the variable for the recovery rate.

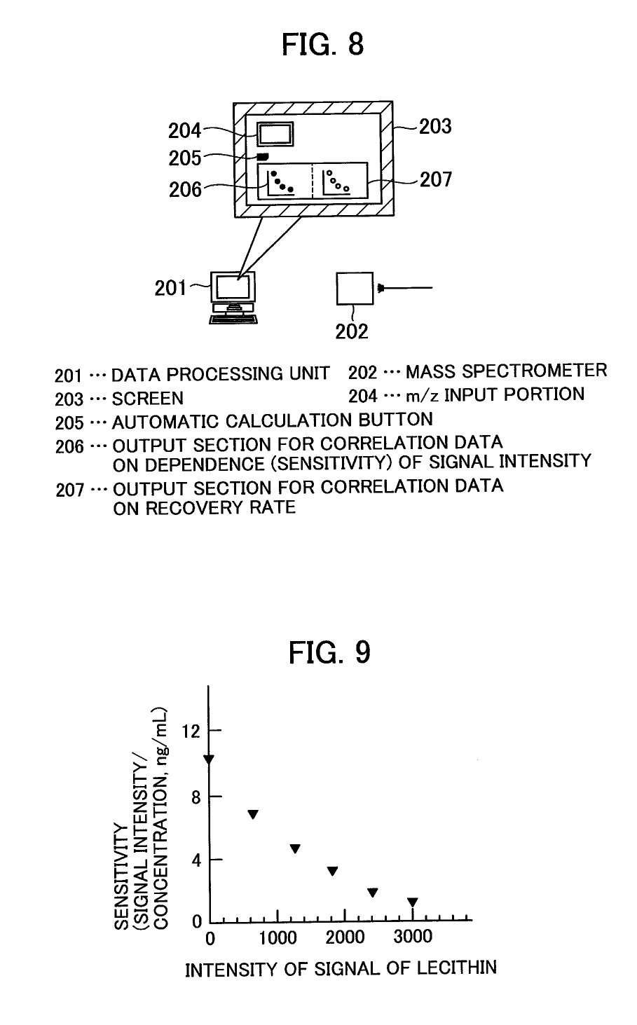

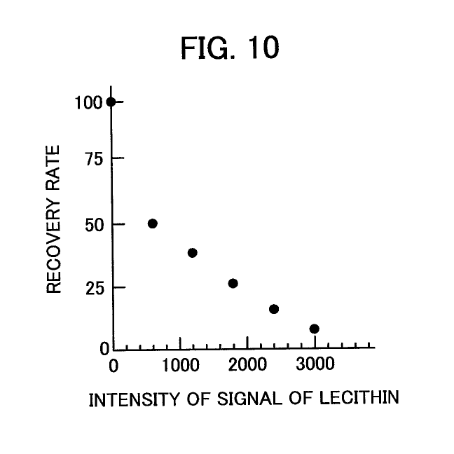

In the second embodiment, the data processing unit 112 has stored therein, in advance, correlation data on the dependence (sensitivity) of the intensities of signals corresponding to mass-to-charge ratios m/z of the analyte substance to be measured with respect to the intensity of a signal corresponding to a mass-to-charge ratio m/z of a substance relating to the property of the matrix, and has stored therein, in advance, the recovery rate of the analyte substance to be measured in the pretreatment. In the present embodiment, the data processing unit 112 has stored therein the dependence (sensitivity) of the intensities of the signals and the recovery rate (FIG. 8), while the intensity of a signal of lecithin, which functions as the substance relating to the property of the matrix, is used as a variable for the recovery rate and the dependence (sensitivity) of the intensities of the signals. The substance relating to the property of the matrix and the analyte substance to be measured are alternately measured at time intervals of several milliseconds to several hundred milliseconds. For example, when the analyte substance to be measured is tacrolimus, which is an immunosuppressant drug, a triple quadrupole mass spectrometer is used as the mass spectrometer 111. The mass-to-charge ratio m/z of the tacrolimus is set to 821.5/768.5 (=Q1:Q3), while the mass-to-charge ratio m/z of the lecithin is set to 787/184 (=Q1:Q3). The ionization is performed to obtain positive ions. The sensitivity of the tacrolimus is calculated from the intensity of the signal of the lecithin contained in the matrix. The concentration of the tacrolimus is calculated from the actual calculated intensity of the signal of the tacrolimus. The concentration of the analyte substance to be measured is calculated from the intensity of the signal of the substance (lecithin in this case) that affects the ion suppression and is contained in the matrix, the intensity of the signal of the analyte substance (tacrolimus in this case) to be measured, and the correlation data stored in the data processing unit 112. When this method is used, the ion suppression can be theoretically corrected without the internal standard. In addition, it is not necessary to construct a calibration curve for each measurement, and the correction can be easily performed.

### Third Embodiment

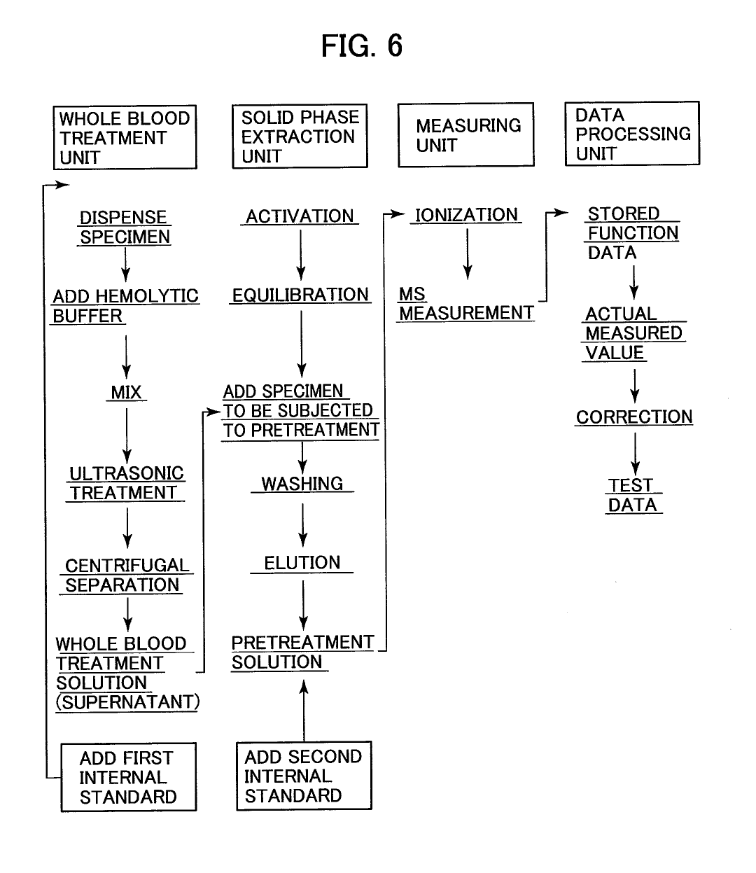

A method for adding two types of internal standards and more accurately calculating the concentration of the analyte substance to be measured is described below. Substances that exhibit ionization efficiencies equal or close to each other are used as the two types of the internal standards. Since the two types of internal standards that exhibit the ionization efficiencies equal or close to each other are used, the recovery rate of the pretreatment device can be directly calculated from measured data. An abnormal condition of the pretreatment device can be alarmed by detecting the difference between the directly calculated recovery rate and the value stored in the database. The difference between the workflow of the measurement in the third embodiment and the workflow of the measurement in the first embodiment is described with reference to FIG. 6, i.e., the difference between a method for adding a first internal standard and that for adding a second internal standard. A patient's specimen of 100 μL, which is dispensed from a blood collection tube, is stored in the whole blood treatment unit 113. The first internal standard, which is stored in the reagent container 104, is dispensed with a volume of 10 μL in each of the cells of the whole blood treatment unit 113 by the rotating arm 106. Before the solution after the pretreatment is transported by the pretreated sample transporting means 109, the second internal standard that is stored in the reagent container 104 is dispensed in the extracted solution receiving mechanism 108 by the rotating arm 105 and added to the treated solution as in the first embodiment. Other processes are the same as those described in the first embodiment. The first internal standard may be dispensed, beforehand, in a blood collection tube that is used to extract the specimen from the patient.

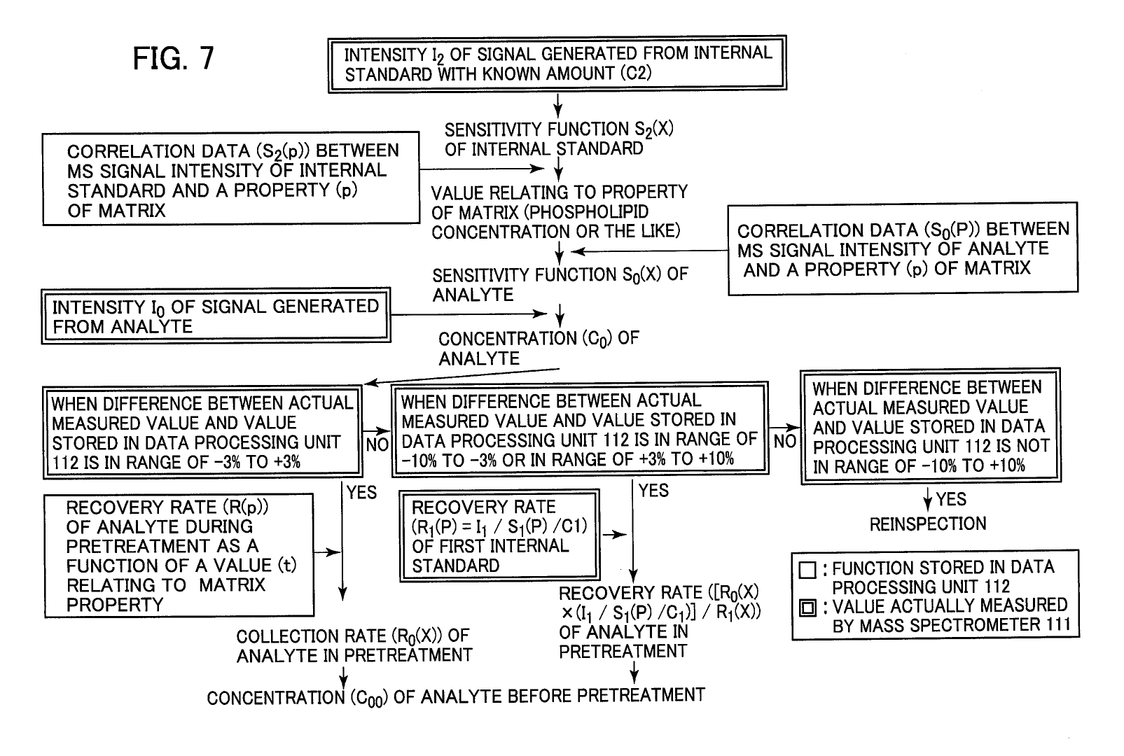

A correction method is described below with reference to FIG. 7. The first internal standard with a known concentration, the second internal standard with a known concentration, and the analyte substance to be measured with a known concentration are added to matrices having different properties in advance. Correlation data is stored in the data processing unit 112. In the correlation data, the value relating to the property of the matrix is plotted along the abscissa, while dependence (sensitivity) of the intensities of signals that correspond to the mass-to-charge ratios m/z of the analyte substance to be measured and the intensities of signals that correspond to the mass-to-charge ratios m/z of the internal standards with respect to the value relating to the property of the matrix, is plotted along the ordinate. The sensitivity is a value obtained by dividing an intensity of a signal corresponding to the mass-to-charge ratios m/z by a concentration. The value relating to the property of the matrix is the concentration of phospholipid. In addition, as values relating to the properties of the matrix, a viscosity, the total mass of proteins and pH may be used. Specifically, a sensitivity function S₁(p) of the first internal standard, a sensitivity function S₂(p) of the second internal standard, and a sensitivity function S₀(p) of the analyte substance to be measured are stored in the data processing unit 112, while the concentration p of the phospholipid is used as a variable for each of the sensitivity functions S₁(p), S₂(p) and S₀(p).

Next, the phospholipid is added to an unknown matrix component (actual sample), so that it contains the first internal standard with a known concentration and the second internal standard with a known concentration. The intensity (actually measured by the mass spectrometer 111) of the signal of the first internal standard is indicated by I₁. The intensity (actually measured by the mass spectrometer 111) of the signal of the second internal standard is indicated by I₂. The intensity (actually measured by the mass spectrometer 111) of the signal of the analyte substance to be measured is indicated by I₀. The value relating to the property of the matrix is indicated by X. The concentration of the first internal standard is known and indicated by C₁. The concentration of the second internal standard is known and indicated by C₂. The concentrations C₁ and C₂ are equal to each other. The sensitivity S₂(X) of the second internal standard is expressed by Equation (1′). The first internal standard and the second internal standard exhibit the ionization efficiencies that are equal or close to each other. Thus, the correlation data on the dependence (sensitivity) of the intensity of the signal corresponding to the mass-to-charge ratio m/z of the first internal standard is the same as that of the second internal standard (Equation (2′)).

S₂(X)=I₂/C₂ (1′)

S₂(p)=S₁(p) (2′)

Next, the recovery rates R₁(p) and R₀(p) of the first internal standard and the analyte substance to be measured are stored in the data processing unit 112 in advance, while the concentration p of the phospholipid in the first internal standard and the analyte substance to be measured is used as a variable for each of the recovery rates R₁(p) and R₀(p). Each of the recovery rates R₁(p) and R₀(p) is a value obtained by dividing the intensity of a signal of a substance after pretreatment by that before treatment; whereby, a signal after pretreatment is obtained by adding a substance to the matrix containing the phospholipid, then by subjecting it to the pretreatment with a resultant concentration p of the phospholipid after the pretreatment, and further subjecting it to the MS detection; and whereby, a signal before pretreatment is obtained by adding a substance to the matrix containing the phospholipid with the concentration p, and directly subjecting it to the MS detection. The value X relating to the property of the matrix can be calculated from the result obtained by Equation (1′) and the function S₂(p) stored in the database in advance. Next, the sensitivity S₀(X) of the analyte substance to be measured is calculated from the value X relating to the property of the matrix and the function S₀(p) stored in the data processing unit 112 beforehand. The concentration C₀ of the analyte substance to be measured is calculated according to Equation (3′) on the basis of the sensitivity S₀(X) and the intensity I₀ (actually measured by the mass spectrometer 111) of the signal of the analyte substance to be measured.

C₀=I₀/S₀(X) (3′)

The ion suppression can be corrected by the aforementioned operations.

The intensity I₁ (actually measured by the mass spectrometer 111) of the signal of the first internal standard is a value in which the recovery rate in the pretreatment and the ionization efficiency are reflected. Note that the sensitivity function S₂(p) is equal to the sensitivity of the first internal standard obtained by plotting the value relating to the property of the matrix along the abscissa, while plotting along the ordinate the dependence (sensitivity) of the intensity of the signal that corresponds to the mass-to-charge ratio m/z of the first internal standard with respect to the value relating to the property of the matrix. Thus, the following Equation (4′) is established, where the recovery rate R₁(p) in the pretreatment is expressed by Equation (4′) using the known concentration C₁, Equation (2′) and the sensitivity function for the first internal standard.

I₁/S₁(p)/C1=R₁(p) (4′)

The left side of Equation (4′) is an actually measured value, while the right side of Equation (4′) is the value stored in the data processing unit 112. In a process of pretreating whole blood, complicated operations such as stirring, an ultrasonic treatment, centrifugal separation and dispensing are performed. Thus, there is a possibility that an error in measured value may occur. In this case, Equation (4′) is not established, and Formula (4″) or (4′″) is established.

I₁/S₁(p)/C1>R₁(p) (4″)

I₁/S₁(p)/C1<R₁(p) (4′″)

In any of the aforementioned cases, a threshold value for the difference between the actual measured value and the value stored in the data processing unit 112 may be preset, so that the concentration of the analyte substance to be measured can be accurately calculated by selecting “the value stored in the data processing unit 112”, “a correction using the actual measured value” or “a retest” (repeating the same analysis again) (FIG. 7). For example, an algorithm is stored in the data processing unit 112 so that: when the difference between the actual measured value and the value stored in the data processing unit 112 is in the range of −3% to +3%, a process proceeds to the selection of “the value stored in the data processing unit 112”; when the difference between the actual measured value and the value stored in the data processing unit 112 is in the range of −10% to −3% or in the range of +3% to +10%, the process proceeds to “the correction using the actual measured value”; and when the difference between the actual measured value and the value stored in the data processing unit 112 is not in the range of −10% to +10%, the process proceeds to “the retest”. Specifically, when the difference is in the range of −3% to +3%, the correction is performed using the value stored in the data processing unit 112, i.e., using the recovery rate R₀(p). The concentration C₀₀ (of the analyte substance to be measured) in the pretreatment is calculated by Equation (5′).

C₀₀=C₀/R₀(X) (5′)

When the difference is in the range of −10% to −3% or in the range of +3% to +10%, the correction is performed using the actual measured value, and the concentration C₀₀ (of the analyte substance to be measured) corrected in the pretreatment is calculated by Equation (5″).

C₀₀=(C₀×R₁(X))/{R₀(X)×(I₁/S₁(p)/C1)} (5″)

When the difference is not in the range of −10% to +10%, the retest is performed.

A user of the instrument can operate a panel to set, in advance, the threshold value for the difference between the actual measured value and the value stored in the data processing unit 112.

### Fourth Embodiment

A method in which a user adds a menu for a new analyte substance to be measured is described below. In a clinical application, dosing plans of a therapeutic drug varies depending on the kind of disease of the patient, the age of the patient, the gender of the patient, the degree of the progress of the disease of the patient, and a clinical institutions (the country and the size of the institutions). Test items may also vary. A dedicated reagent (antibody reagent) is required for each of drugs in a conventional immunoassay. In a method in which the mass spectrometer (MS) is used as a detector, an analyte substance to be measured is selected on the basis of the mass-to-charge ratio m/z (mass/charges) of the analyte substance to be measured. Thus, the method is well suited for measurement of multiple items. Specifically, an analyte substance to be measured can be flexibly added, while an analyte substance to be measured can be flexibly removed. Especially, in the correction method according to the present invention, it is not necessary to construct a calibration curve for each tests using a stable-isotope-substituted analyte molecule for the analyte substance to be measured as the internal standard, unlike the conventional method, and it is easy to add an analyte substance to be measured and remove an analyte substance to be measured. The correlation data on the dependence (sensitivity) of the intensities of signals corresponding to the mass-to-charge ratios m/z of a plurality of analyte substances to be measured and at least one of the internal standards with respect to the value relating to each property of the matrix, and the recovery rates of the analyte substances to be measured for the matrix, are stored in the data processing unit 201. The concentrations of the analyte substances to be measured are calculated on the basis of the intensities of the signals of the analyte substances to be measured and the intensity of a signal of at least one of the internal standards. An analyte substance to be measured can be newly added by registering the following three information pieces on the internal standards and the analyte substances to be measured, in addition to the information that has already been registered in the data processing unit 201. The three information pieces are the correlation data on the dependence (sensitivity) of the intensities of signals corresponding to the mass-to-charge ratios m/z of the analyte substance to be measured and newly added with respect to the value relating to each property of the matrix; the recovery rate of the newly-added analyte substance for the matrix; and the mass-to-charge ratio m/z of the substance to be newly added. In principle, a plurality of substances to be measured can be added. The three information pieces can be obtained by an automatic measurement performed by a mass spectrometer 202. Specifically, the mass spectrometer 202 performs, in advance, an infusion measurement on the analyte substance to be measured that is to be newly added and obtains data of the mass-to-charge ratio m/z of the substance to be newly added. Next, the mass-to-charge ratio m/z of the analyte substance to be measured is input to an m/z input portion 204 on a screen of a data processing unit 201. When an automatic calculation button 205 is clicked, the mass spectrometer 111 performs an actual measurement while a parameter is changed. The data processing unit 112 calculates, on the basis of the measured data, conditions (an ionization condition, a fragmentation voltage, and the like) necessary for mass spectrometry to be performed on the analyte substance to be measured. After that, the correlation data on the dependence (sensitivity) of the intensities of signals that correspond to the mass-to-charge ratios m/z of the newly-added analyte substance to be measured with respect to the value relating to each of the properties of the matrix, is output to an output section 206. In addition, the recovery rate of the newly-added analyte substance to be measured for the matrix is calculated and output to an output section 207. All information (all data for the signal intensities corresponding to mass-to-charge ratios m/z detected at the respective times) that is obtained in this process, is stored and can be retrieved when necessary. That is, if a failure of an analysis occurs, data of the set conditions can be reviewed and modified. Thus, the time, cost and effort for resetting the conditions are reduced. The analyte substance to be measured is newly added in the aforementioned manner, so that a plurality of analyte substances to be measured can be automatically added, in principle, regardless of the type of an internal standard.

### DESCRIPTION OF THE REFERENCE NUMERALS

101 Solid phase extraction cartridge
102 Cartridge holding vessel
103 Cartridge transporting means
104 Reagent container
105, 106 Rotating arm
107 Pressure applying unit
108 Extracted solution receiving mechanism
109 Pretreated sample transporting means
110 Ionizer
111 Mass spectrometer
112 Data processing unit
113 Whole blood treatment unit

1. An analyzer comprising,
a pretreatment device that includes a solid phase extraction mechanism,
a mass spectrometer that performs mass spectrometry on a sample pretreated by the pretreatment device and then subjected to ionization, the analyzer further comprising:
a storage unit that stores data on the concentration of a substance inhibiting ionization in the sample, data on dependence of signal intensities of a analyte substance to be measured and an internal standard, and data on a recovery rate; and
a correcting unit that corrects measurement results of the sample and the internal standard on the basis of the data stored in the storage unit.

2. The analyzer according to claim 1,
wherein a substance that inhibits the ionization is phospholipid.

3. The analyzer according to claim 2,
wherein the phospholipid is at least one of glycerophospholipid and sphingophospholipid.

4. The analyzer according to claim 3,
wherein the glycerophospholipid is at least one selected from a group consisting of lecithin (phosphatidylcholine), lysolecithin and cephalin (phosphatidylethanolamine).

5. The analyzer according to claim 3,
wherein the sphingophospholipid is sphingomyelin.

6. An analyzer including a pretreatment device that includes a solid phase extraction mechanism, and a mass spectrometer that performs mass spectrometry on a sample pretreated by the pretreatment device and then subjected to ionization, the analyzer comprising:
a storage unit that stores at least one of a viscosity of the sample, the total mass of proteins of the sample, and pH of the sample and stores data on dependence of signal intensities of an analyte substance to be measured and an internal standard, and data on a recovery rate; and
a correcting unit that corrects measurement results of the sample and the internal standard on the basis of the data stored in the storage unit.

7. An analyzer comprising:
a pretreatment device;
pretreated sample transporting means;
an ionizer that ionizes a sample and causes the sample to be introduced in a mass spectrometer;
the mass spectrometer that is capable of performing an analysis and measurement; and
a data processing unit,
wherein the pretreatment device includes:
a solid phase extraction cartridge;
a cartridge holding vessel that holds a solid phase extraction cartridge therein;
cartridge transporting means that is capable of holding a plurality of storage sections (cartridge holding vessels) and has a continuous-track;
a whole blood treatment unit that is capable of performing a purifying treatment on whole blood;
a reagent container that is capable of storing a plurality of reagents;
a rotating arm that is capable of transporting a reagent from the reagent container to the solid phase extraction cartridge;
a rotating arm that is capable of transporting a reagent from the reagent container to the whole blood treatment unit;
a pressure applying unit that is capable of continuously and randomly applying pressure to the insides of the storage sections; and
an extracted solution receiving mechanism that selectively receives a solution extracted from the separating agent stored in the storage sections,
wherein at least one internal standard is used, and the data processing unit stores correlation data on dependence (sensitivity) of the intensities of signals that indicate mass-to-charge ratios m/z of an analyte substance to be measured and the internal standard with respect to the matrix containing phospholipid with the different concentration, and stores a recovery rate of the analyte substance to be measured for the matrix, and
wherein the concentrations of the phospholipid and the concentration of the analyte substance to be measured are calculated from the signal intensities of the analyte substance to be measured and the internal standard, the intensities being actually measured by the mass spectrometer.

8. The analyzer according to claim 7,
wherein the phospholipid is at least one of lecithin (phosphatidylcholine), lysolecithin, cephalin (phosphatidylethanolamine) and sphingomyelin.

9. The analyzer according to claim 7, further comprising:
a first internal standard adding mechanism that adds the at least one internal standard to a specimen transported to the whole blood treatment unit; and
a second internal standard adding mechanism that adds the at least one internal standard to the extracted solution dispensed in the extracted solution receiving mechanism.

10. The analyzer according to claim 7,
wherein the internal standard is a stable-isotope-substituted analyte molecule for the analyte substance to be measured or is a pseudo compound.

11. The analyzer according to claim 1,
wherein the extraction mechanism of the pretreatment device is a liquid-liquid extraction mechanism.

12. The analyzer according to claim 1,
wherein the extraction mechanism of the pretreatment device is a deproteinization mechanism.

13. The analyzer according to claim 1,
wherein the extraction mechanism of the pretreatment device is an ultrafiltration membrane mechanism.

14. The analyzer according to claim 1,
wherein the extraction mechanism of the pretreatment device uses antibody magnetic beads.

15. The analyzer according to claim 1,
wherein the whole blood treatment unit includes an ultrasonic wave generating mechanism, centrifugal separation mechanism, a stirring mechanism and a solution dispensing mechanism that is capable of dispensing a treated solution in the solid phase extraction cartridge.

16. The analyzer according to claim 15,
wherein the ultrasonic wave generating mechanism has a temperature control function.
